(19) United States
(12) Patent Application Publication
(10) Pub. No.: US 2025/0228445 A1
(43) Pub. Date: Jul. 17, 2025

(54) MEDICAL OBSERVATION SYSTEM AND LIGHT SOURCE CONTROL DEVICE

(71) Applicant: Sony Olympus Medical Solutions Inc., Tokyo (JP)

(72) Inventor: Yuki OGAWA, Tokyo (JP)

(73) Assignee: Sony Olympus Medical Solutions Inc., Tokyo (JP)

(21) Appl. No.: 19/008,755

(22) Filed: Jan. 3, 2025

(30) Foreign Application Priority Data
Jan. 16, 2024 (JP) 2024-004758

(51) Int. Cl. A61B 1/06; A61B 1/05; A61B 90/20; G02B 21/00

(52) U.S. Cl. CPC A61B 1/0655; A61B 1/05; A61B 90/20; G02B 21/0012

(57) ABSTRACT

A medical observation system includes: a light source device configured to emit first light and second light to an observation target; an imaging device configured to image return light of the first light and the second light from the observation target, and change number of horizontal lines to be simultaneously exposed by at least one of the return light of the first light and the return light of the second light; and a light source controller configured to adjust light emission periods of the first light and the second light such that energy of light obtained by exposing the return light of the first light in the imaging device and energy of light obtained by exposing the return light of the second light in the imaging device have a predetermined ratio.

### CROSS-REFERENCE TO RELATED APPLICATION

This application claims priority from Japanese Application No. 2024-004758, filed on Jan. 16, 2024, the contents of which are incorporated by reference herein in its entirety.

### BACKGROUND

The present disclosure relates to a medical observation system and a light source control device.

In the related art, there has been known a medical observation system that images an observation target in a living body or the like using a complementary metal oxide semiconductor (CMOS), which is a rolling shutter type imaging element, and observes the observation target (See, for example, JP 2022-082350 A).

In the medical observation system described in JP 2022-082350 A, a normal light image and a fluorescence image are generated. Specifically, the normal light image is an image obtained by irradiating the observation target with normal light in a visible wavelength band from the light source device and imaging the normal light through the observation target. In addition, the fluorescence image is an image obtained by irradiating the observation target with excitation light from the light source device and imaging the fluorescence from the observation target excited by the excitation light.

Here, in the medical observation system described in JP 2022-082350 A, in order to generate a bright fluorescence image, excitation light is emitted even outside the entire line exposure period in which all horizontal lines of the effective pixel region in the imaging element are simultaneously exposed.

### SUMMARY

In the medical observation system described in JP 2022-082350 A, in a case where PWM control is performed in controlling the operation of the light source device, a light emission period of the normal light changes within the entire line exposure period, whereas a light emission period of the excitation light changes even outside the entire line exposure period. Therefore, in a case where the light emission period of the excitation light is changed at the same rate as the change in the light emission period of the normal light, a ratio between brightness of the normal light image and brightness of the fluorescence image is changed, and a balance of the brightness is deteriorated. That is, there is a problem that it is difficult to generate an image suitable for observation.

According to one aspect of the present disclosure, there is provided a medical observation system including: a light source device configured to emit first light and second light to an observation target; an imaging device configured to image return light of the first light and the second light from the observation target, and change number of horizontal lines to be simultaneously exposed by at least one of the return light of the first light and the return light of the second light; and a light source controller configured to adjust light emission periods of the first light and the second light such that energy of light obtained by exposing the return light of the first light in the imaging device and energy of light obtained by exposing the return light of the second light in the imaging device have a predetermined ratio.

According to another aspect of the present disclosure, there is provided a medical observation system including: a light source device configured to emit light to an observation target; an imaging device configured to image return light of the light from the observation target, and change number of horizontal lines to be simultaneously exposed by the return light of the light; and a light source controller configured to adjust a light emission period of the light based on an energy index value serving as an index of a target value of energy of the return light exposed in the imaging device.

According to still another aspect of the present disclosure, there is provided a light source control device including a light source controller configured to control an operation of a light source device, and cause the light source device to emit first light and second light to an observation target, wherein each return light of the first light and the second light from the observation target is imaged by an imaging device that changes the number of horizontal lines simultaneously exposed by at least one of the return light of the first light and the return light of the second light, and the light source controller is configured to adjust light emission periods of the first light and the second light such that energy of light obtained by exposing the return light of the first light in the imaging device and energy of light obtained by exposing the return light of the second light in the imaging device have a predetermined ratio.

According to yet another aspect of the present disclosure, there is provided a light source control device including a light source controller is configured to control an operation of a light source device, and cause the light source device to emit light to an observation target, wherein return light of the light from the observation target is imaged by an imaging device that changes the number of horizontal lines simultaneously exposed by the return light, and the light source controller is configured to adjust a light emission period of the light based on an energy index value serving as an index of a target value of energy of the return light exposed in the imaging device.

### BRIEF DESCRIPTION OF THE DRAWINGS

FIG. 1 is a diagram illustrating a configuration of a medical observation system according to an embodiment;

FIG. 2 is a block diagram illustrating a configuration of a camera head and a control device;

FIG. 3 is a diagram for describing a problem of the present disclosure;

FIG. 4 is a diagram for describing a problem of the present disclosure;

FIG. 5 is a diagram for describing a problem of the present disclosure;

FIG. 6 is a diagram for describing a problem of the present disclosure;

FIG. 7 is a diagram for describing a problem of the present disclosure;

FIGS. 8A and 8B are diagrams for describing a problem of the present disclosure;

FIG. 9 is a diagram for describing a calculation expression of an exposure area in a first imaging element;

FIG. 10 is a diagram for describing a calculation expression of an exposure area in a second imaging element;

FIG. 11 is a diagram for describing a fourth modification of the embodiment;

FIG. 12 is a diagram for describing a fifth modification of the embodiment;

FIG. 13 is a diagram for describing a seventh modification of the embodiment;

FIG. 14 is a diagram for describing seventh modification of the embodiment;

FIG. 15 is a diagram for describing a twelfth modification of the embodiment;

FIG. 16 is a diagram for describing a thirteenth modification of the embodiment;

FIG. 17 is a diagram for describing a fourteenth modification of the embodiment; and

FIG. 18 is a diagram for describing a fourteenth modification of the embodiment.

### DETAILED DESCRIPTION

Hereinafter, modes for carrying out the present disclosure (embodiments) will be described with reference to the drawings. Note that the present disclosure is not limited by the embodiments described below. Furthermore, in the description of the drawings, the same portions are denoted by the same reference numerals.

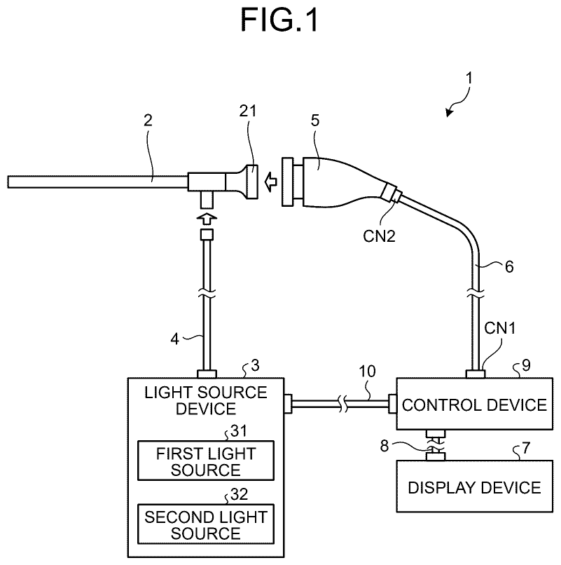

Configuration of Medical Observation System FIG. 1 is a diagram illustrating a configuration of a medical observation system 1 according to an embodiment.

In the present embodiment, the medical observation system 1 is a medical endoscope system that observes an observation target (in a living body) using an endoscope. As illustrated in FIG. 1, the medical observation system 1 includes an insertion unit 2, a light source device 3, a light guide 4, a camera head 5, a first transmission cable 6, a display device 7, a second transmission cable 8, a control device 9, and a third transmission cable 10.

In the present embodiment, the insertion unit 2 includes a rigid endoscope. That is, the insertion unit 2 has an elongated shape in which the entire insertion unit 2 is rigid, or a part of the insertion unit 2 is soft and the other part of the insertion unit 2 is rigid, and is inserted into the observation target (in the living body). An optical system configured using one or a plurality of lenses and condensing light from the observation target is provided in the insertion unit 2.

The light source device 3 is connected to one end of the light guide 4, and supplies light to the one end of the light guide 4 under the control of the control device 9.

As illustrated in FIG. 1, the light source device 3 includes a first light source 31 and a second light source 32.

The first light source 31 emits first light (wideband light such as white light, or narrowband light such as red light, green light, or blue light) including at least a part of a visible light wavelength band. Examples of the configuration of the first light source 31 include a configuration including a white light emitting diode (LED), and a configuration including three light sources that respectively emit red light, green light, and blue light, and an optical element that synthesizes the red light, the green light, and the blue light. The first light source 31 may be constructed with an LED or a semiconductor laser. The number of first light sources 31 may be one or more.

The second light source 32 emits excitation light for exciting a substance included in the observation target. The excitation light corresponds to second light according to the present disclosure. The second light source 32 may be constructed with an LED or a semiconductor laser. The number of second light sources 32 may be one or more. Furthermore, the second light source 32 may emit, for example, infrared light or ultraviolet light.

Here, examples of the substance included in the observation target excited by the excitation light include a drug or a fluorescent dye applied to the observation target, or a fluorescent substance derived from the observation target constituting the observation target itself.

Examples of the above-described drug to be applied to the observation target include “5-ALA (PP-IX)”, “ADS780WS”, “ADS830WS”, “aggregation-induced emission dots allophycocyanin (APC)”, “boron-dipyrromethane (BODIPY)”, “CLR 1502”, “Flavins”, “fluorescamine”, “fluorescein”, “fluoro-gold”, “green fluorescence protein”, “ICG (indocyanine green)”, “IRDye 78”, “IR-PEG nanoparticles”, “Isothiocyanate”, “rose Bengal”, “SGM-101”, and “trypan blue”.

In addition, the above-described fluorescent dyes to be applied to the observation target include “coumarine”, “Cy3”, “DyLight547”, “GE3126”, “metal nanoclusters”, “oxacarbocyanine”, “Rhodamine”, “Riboflavin”, “fluorescein”, “AlexaFluor660”, “AlexaFluor680”, “AlexaFluor700”, “Cy5”, “Cy5.5”, “Dy677”, “Dy682”, “Dy752”, “DyLight647”, “HiLyte Fluor 647”, “HiLyte Fluor 680”, “IRDye 700DX”, “methylene blue”, “Porphyrins”, “Porphysomes”, “VivoTag-680”, “VivoTag-s680”, “AlexaFluor750”, “AlexaFluor790”, “carbocyanine”, “conjugated copolymers”, “CW800-CA”, “Cy7”, “Cy7.5”, “cyanine dyes”, “Dy780”, “HiLyte Fluor 750”, “Indocarbocyanine”, “IR-786”, “IRDye 800CW”, “IRDye 800RS”, “IRDye 800BK”, “Nervelight”, “OTL-38”, “Polymethine”, “VivoTag-S750”, “ASP5354”, and “Xanthene”.

Furthermore, examples of the fluorescent substance derived from the observation target constituting the observation target itself include “collagen”, “elastin”, and “NADH”.

In the present embodiment, the light source device 3 is configured separately from the control device 9, but the present disclosure is not limited thereto, and a configuration provided in the same casing as that of the control device 9 may be adopted.

One end of the light guide 4 is detachably connected to the light source device 3, and the other end thereof is detachably connected to the insertion unit 2. Then, the light guide 4 transmits light (first light or excitation light) supplied from the light source device 3 from one end to the other end, and supplies the light to the insertion unit 2. The light (first light or excitation light) supplied to the insertion unit 2 is emitted from the distal end of the insertion unit 2 and applied to the observation target. When the observation target is irradiated with the first light, return light of the first light from the observation target (reflected light of the first light) is condensed by the optical system in the insertion unit 2. In addition, when the observation target is irradiated with the excitation light, return light of the excitation light from the observation target is condensed by the optical system in the insertion unit 2. The return light of the excitation light includes, in addition to the excitation light reflected by the observation target, fluorescence emitted from a substance contained in the observation target when the observation target is irradiated with the excitation light and the substance is excited.

Hereinafter, for convenience of description, the return light of the first light from the observation target described above is described as first return light, and the return light of the excitation light from the observation target is described as second return light.

The camera head 5 corresponds to an imaging device according to the present disclosure. The camera head 5 is detachably connected to an eyepiece unit 21 of the insertion unit 2. Then, under the control of the control device 9, the camera head 5 images the first return light and the second return light condensed by the insertion unit 2 to generate a pixel signal. Hereinafter, for convenience of description, the pixel signal is referred to as a captured image.

Note that a detailed configuration of the camera head 5 will be described in “Configuration of Camera Head” described later.

One end of the first transmission cable 6 is detachably connected to the control device 9 via a connector CN1 (FIG. 1), and the other end thereof is detachably connected to the camera head 5 via a connector CN2 (FIG. 1). Then, the first transmission cable 6 transmits the captured image and the like output from the camera head 5 to the control device 9, and transmits a control signal, a synchronization signal, a clock, power, and the like output from the control device 9 to the camera head 5.

Note that, in the transmission of the captured image and the like from the camera head 5 to the control device 9 via the first transmission cable 6, the captured image and the like may be transmitted as an optical signal or may be transmitted as an electric signal. The same applies to transmission of a control signal, a synchronization signal, and a clock from the control device 9 to the camera head 5 via the first transmission cable 6.

The display device 7 includes a display using liquid crystal, organic electro luminescence (EL), or the like, and displays an image based on a video signal from the control device 9 under the control of the control device 9.

One end of the second transmission cable 8 is detachably connected to the display device 7, and the other end thereof is detachably connected to the control device 9. Then, the second transmission cable 8 transmits the video signal processed by the control device 9 to the display device 7.

The control device 9 corresponds to a light source control device according to the present disclosure. The control device 9 includes a central processing unit (CPU), a field-programmable gate array (FPGA), and the like, and integrally controls operations of the light source device 3, the camera head 5, and the display device 7.

Note that a detailed configuration of the control device 9 will be described in “Configuration of Control Device” described later.

One end of the third transmission cable 10 is detachably connected to the light source device 3, and the other end thereof is detachably connected to the control device 9. Then, the third transmission cable 10 transmits the control signal from the control device 9 to the light source device 3.

### Configuration of Camera Head

Next, a configuration of the camera head 5 will be described.

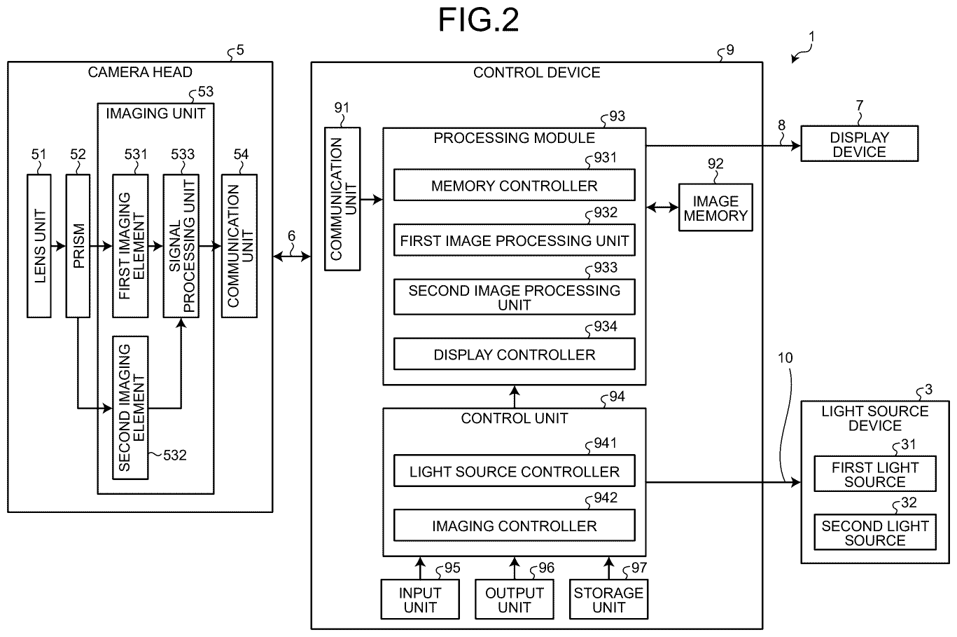

FIG. 2 is a block diagram illustrating configurations of the camera head 5 and the control device 9.

As illustrated in FIG. 2, the camera head 5 includes a lens unit 51, a prism 52, an imaging unit 53, and a communication unit 54.

The lens unit 51 includes one or a plurality of lenses. Then, the lens unit 51 forms images of the first return light and second return light (subject image) condensed by the insertion unit 2 on imaging surfaces of the first and second imaging elements 531 and 532, respectively.

The prism 52 separates the first return light and second return light (subject images) via the lens unit 51 into light of first and second wavelength bands different from each other. The light of the first wavelength band is light of a wavelength band excluding the wavelength band of the second return light, and is light including the wavelength band of visible light that is the first return light. Hereinafter, the light in the first wavelength band will be referred to as a first subject image. The light of the second wavelength band is light of a wavelength band excluding the wavelength band of visible light, and is light including the wavelength band of the second return light. Hereinafter, the light in the second wavelength band will be referred to as a second subject image. Then, the prism 52 advances the first subject image (first return light) toward the first imaging element 531. Furthermore, the prism 52 advances the second subject image (second return light) toward the second imaging element 532.

The imaging unit 53 images the observation target under the control of the control device 9. As illustrated in FIG. 2, the imaging unit 53 includes the first imaging element 531, the second imaging element 532, and a signal processing unit 533.

The first and second imaging elements 531 and 532 receive the subject image and convert the subject image into an electric signal (analog signal). In the present embodiment, each of the first and second imaging elements 531 and 532 includes a CMOS that is a rolling shutter type imaging element in which a plurality of pixels is two-dimensionally arranged in units of horizontal lines.

Here, although not specifically illustrated, the first imaging element 531 includes an invalid region that is not electrically guaranteed, an optical black region (OB region), and an effective pixel region that converts the first subject image formed by the lens unit 51 into a pixel signal and outputs the pixel signal. Note that the second imaging element 532 similarly includes an invalid region, an optical black region (OB region), and an effective pixel region.

Then, the first imaging element 531 images the first subject image (first return light) through the prism 52 under the control of the control device 9. That is, the first imaging element 531 images light including at least a part of the visible light wavelength band. Hereinafter, for convenience of description, the captured image generated by imaging the first subject image by the first imaging element 531 will be referred to as a normal light image.

Furthermore, the second imaging element 532 images the second subject image via the prism 52 under the control of the control device 9. That is, the second imaging element 532 images light including at least a part of the invisible light wavelength band. Hereinafter, for convenience of description, the captured image generated by imaging the second subject image by the second imaging element 532 will be referred to as a fluorescence image.

Note that the number of pixels of the normal light image and the number of pixels of the fluorescence image may be different from each other or may be the same.

Under the control of the control device 9, the signal processing unit 533 performs signal processing on the captured images (analog signals) generated by the first and second imaging elements 531 and 532 and outputs the captured images (digital signals).

For example, the signal processing unit 533 performs a process of removing reset noise, a process of multiplying an analog gain for amplifying the analog signal, and a signal process such as A/D conversion on the captured images (analog signals) generated by the first and second imaging elements 531 and 532.

The communication unit 54 functions as a transmitter that transmits the captured images sequentially output from the imaging unit 53 to the control device 9 via the first transmission cable 6. The communication unit 54 includes, for example, a high-speed serial interface that communicates the captured image with the control device 9 via the first transmission cable 6 at a transmission rate of 1 Gbps or more.

Note that the communication unit 54 may alternately transmit the normal light image and the fluorescence image to the control device 9, or may simultaneously transmit the images.

### Configuration of Control Device

Next, a configuration of the control device 9 will be described with reference to FIG. 2.

As illustrated in FIG. 2, the control device 9 includes a communication unit 91, an image memory 92, a processing module 93, a control unit 94, an input unit 95, an output unit 96, and a storage unit 97.

The communication unit 91 functions as a receiver that receives the captured images sequentially transmitted from the camera head 5 (communication unit 54) via the first transmission cable 6. The communication unit 91 includes, for example, a high-speed serial interface that communicates the captured images with the communication unit 54 at a transmission rate of 1 Gbps or more.

The image memory 92 includes, for example, a dynamic random access memory (DRAM) or the like. The image memory 92 can temporarily store a plurality of frames of captured images sequentially output from the camera head 5 (communication unit 54).

The processing module 93 processes the captured image sequentially transmitted from the camera head 5 (communication unit 54) and received by the communication unit 91 under the control of the control unit 94. As illustrated in FIG. 2, the processing module 93 includes a memory controller 931, a first image processing unit 932, a second image processing unit 933, and a display controller 934.

The memory controller 931 controls writing of a captured image into the image memory 92 and reading of the captured image from the image memory 92. More specifically, the memory controller 931 writes the normal light image received by the communication unit 91 in the image memory 92, reads the normal light image from the image memory 92 at a specific timing, and inputs the normal light image to the first image processing unit 932. In addition, the memory controller 931 writes the fluorescence image received by the communication unit 91 in the image memory 92, reads the fluorescence image from the image memory 92 at a specific timing, and inputs the fluorescence image to the second image processing unit 933.

The first image processing unit 932 executes first image processing on the input normal light image.

Examples of the first image processing include optical black subtraction processing (clamp processing), white balance adjustment processing, demosaic processing, color correction matrix processing, gamma correction processing, YC processing of converting an RGB signal into a luminance chrominance signal (Y, Cb/Cr SIGNAL), gain adjustment, noise removal, filter processing of enhancing a structure, and the like.

The second image processing unit 933 executes second image processing on the input fluorescence image.

Examples of the second image processing include optical black subtraction processing (clamp processing), white balance adjustment processing, demosaic processing, color correction matrix processing, gamma correction processing, YC processing of converting an RGB signal into a luminance chrominance signal (Y, Cb/Cr SIGNAL), gain adjustment, noise removal, filter processing of enhancing a structure, and the like.

Note that the first and second image processing may be different from each other, or may be the same image processing.

Under the control of the control unit 94, the display controller 934 generates a video signal for displaying the normal light image after the first image processing is executed by the first image processing unit 932 and the fluorescence image after the second image processing is executed by the second image processing unit 933. Then, the display controller 934 outputs the video signal to the display device 7 via the second transmission cable 8.

The control unit 94 is realized by executing various programs stored in the storage unit 97 by a controller such as a CPU or a micro processing unit (MPU), and controls the operations of the light source device 3, the camera head 5, and the display device 7 and controls the entire operation of the control device 9. Note that the control unit 94 is not limited to the CPU or the MPU, and may be configured by an integrated circuit such as an application specific integrated circuit (ASIC) or an FPGA. As illustrated in FIG. 2, the control unit 94 has functions as a light source controller 941 and an imaging controller 942. Note that the light source controller 941 is not limited to the configuration provided in the control device 9, and a configuration provided in the light source device 3 may be adopted.

Note that the function of the imaging controller 942 will be described in “Problem of Present Disclosure” described later. Furthermore, the function of the light source controller 941 will be described in “Problem of Present Disclosure” and “Function of Light Source Controller” described later.

The input unit 95 is configured using an operation device such as a mouse, a keyboard, and a touch panel, and receives a user operation by a user such as an operator.

Then, the input unit 95 outputs an operation signal corresponding to the user operation to the control unit 94.

The output unit 96 is configured using a speaker, a printer, or the like, and outputs various types of information.

The storage unit 97 stores a program executed by the control unit 94, information necessary for processing of the control unit 94, and the like.

### Problems of Present Disclosure

Before describing the operation of the control device 9, the problem of the present disclosure will be described.

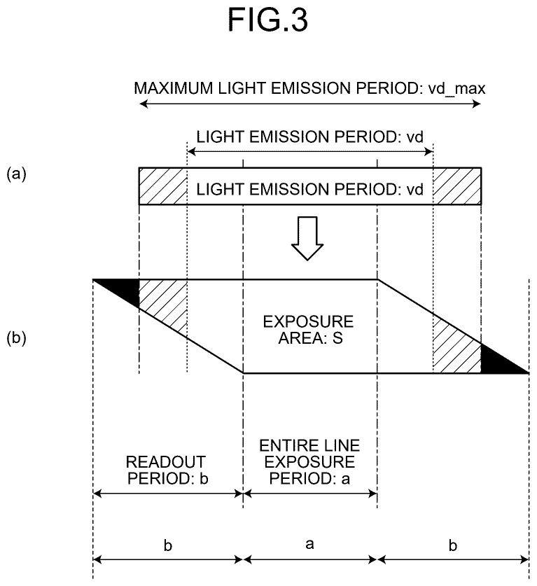

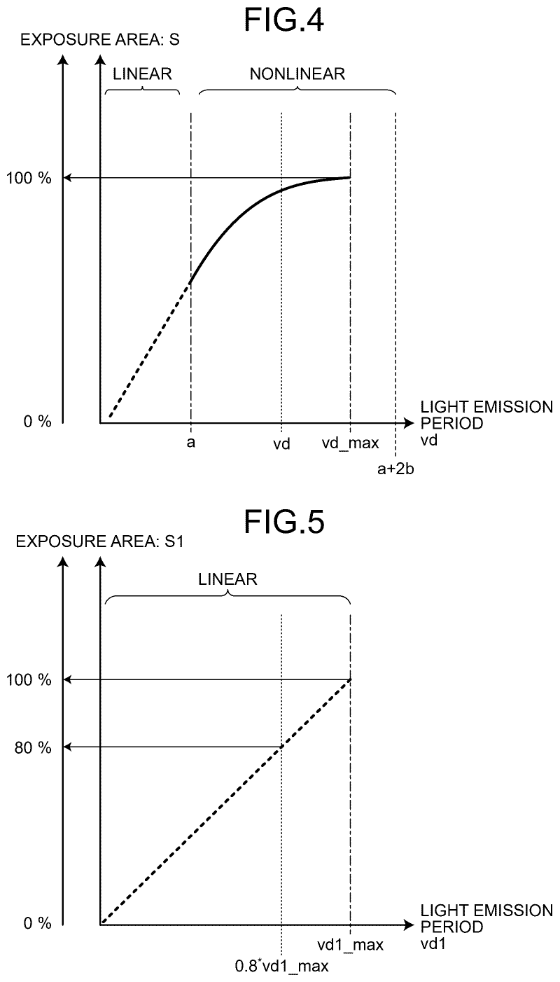

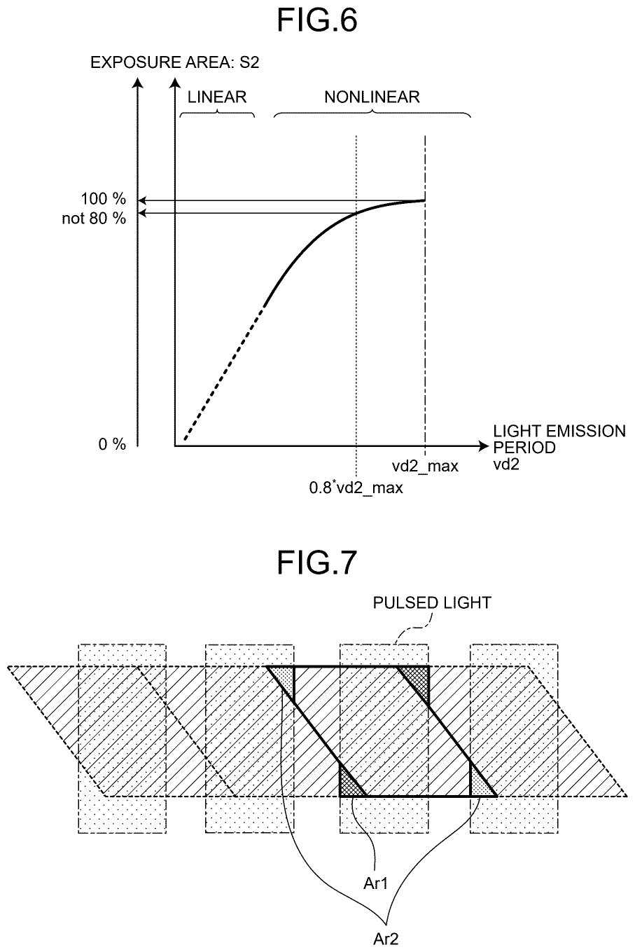

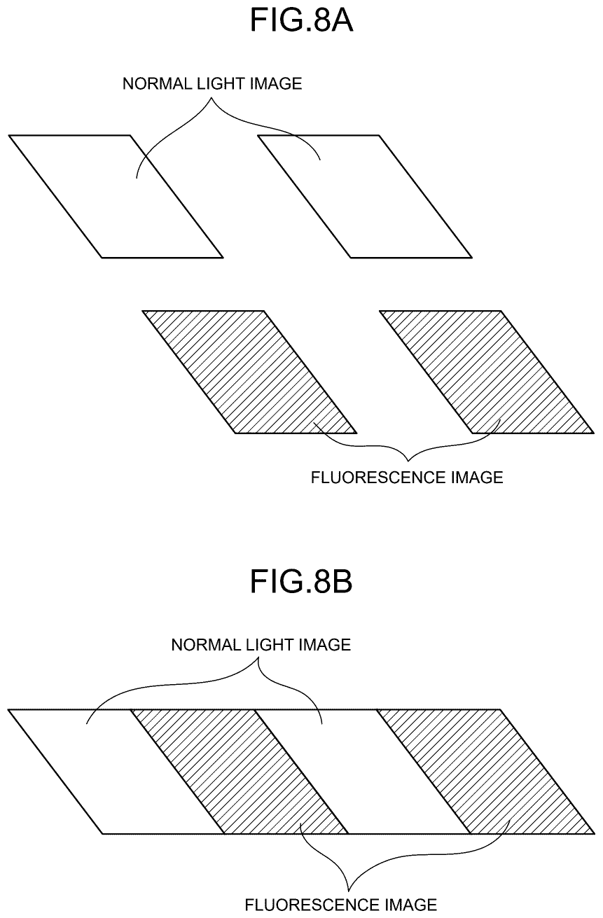

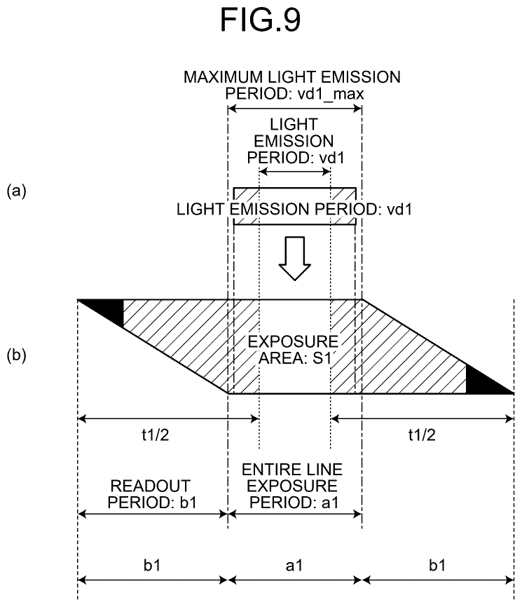

FIGS. 3 to 8B are diagrams for explaining the problem of the present disclosure. Specifically, in (b) of FIG. 3, a vertical axis represents the horizontal line of the rolling shutter type imaging element (first and second imaging elements 531 and 532) (the uppermost line indicates the uppermost horizontal line (the first horizontal line), and the lowermost line indicates the lowermost horizontal line (the last line)), and a horizontal axis represents time. A parallelogram region is a region that contributes to generation of a captured image in one field (one frame). (a) of FIG. 3 is a diagram illustrating a light emission period (light emission width) vd of the light emitted from the light source (first and second light sources 31 and 32). FIG. 4 is a diagram illustrating a relationship between an exposure area S and a light emission period vd (exposure period) of light emitted from a light source (first and second light sources 31 and 32) in the imaging element. Here, the exposure area S corresponds to an “energy index value” according to the present disclosure, and is a value obtained by adding the exposure periods of the exposed horizontal lines. FIG. 5 is a diagram corresponding to FIG. 4, and is a diagram illustrating a relationship between the exposure area S (hereinafter, referred to as an exposure area S1) in the first imaging element 531 and the light emission period vd (hereinafter, referred to as a light emission period vd1) of the first light emitted from the first light source 31. FIG. 6 is a diagram corresponding to FIG. 4, and is a diagram illustrating the relationship between the exposure area S (hereinafter, referred to as an exposure area S2) in the second imaging element 532 and the light emission period vd (hereinafter, referred to as a light emission period vd2) of the excitation light emitted from the second light source 32. In FIG. 7, a parallelogram indicated by a broken line and a solid line corresponds to (b) of FIG. 3. Furthermore, in FIG. 7, a rectangle indicated by an alternate long and short dash line represents light (pulsed light) emitted from a light source (first and second light sources 31 and 32). FIGS. 8A and 8B are diagrams corresponding to (b) of FIG. 3, and an outlined parallelogram indicates one field (one frame) contributing to generation of the normal light image. In addition, a slanted parallelogram indicates one field (one frame) contributing to generation of a fluorescence image. That is, the upper part of FIG. 8A illustrates one field (one frame) that contributes to generation of the normal light image by the first imaging element 531. Furthermore, the lower part of FIG. 8A illustrates one field (one frame) that contributes to generation of a fluorescence image by the second imaging element 532. FIG. 9 illustrates one field (one frame) contributing to generation of a normal light image and a fluorescence image in a case where only one imaging element according to the present disclosure is provided.

The imaging controller 942 sequentially starts exposure of the first and second imaging elements 531 and 532 in one field period for each horizontal line, and performs so-called rolling shutter type imaging control of sequentially performing reading for each horizontal line for which a predetermined period (so-called shutter speed) has elapsed from the start of exposure.

Here, in a case where the rolling shutter type imaging element is adopted, when the light emission period vd is changed within the entire line exposure period a, the relationship between the exposure area S and the light emission period vd becomes linear as illustrated in FIG. 4. Meanwhile, when the light emission period vd is changed outside the entire line exposure period a, the relationship between the exposure area S and the light emission period vd becomes nonlinear. The non-linearity means that the number of horizontal lines exposed simultaneously changes. In the present embodiment, as described above, since the first and second imaging elements 531 and 532 are rolling shutter type imaging elements, the first and second imaging elements has a region in which the relationship between the exposure area S and the light emission period vd is non-linear.

In the present embodiment, for the first light source 31, the light source controller 941 executes PWM control for changing the light emission period vd1 of the first light in a state where the maximum light emission period vd max (hereinafter, it is described as a maximum light emission period vd1_max) of the first light is within the entire line exposure period a (hereinafter, it is described as an entire line exposure period a1) in the first imaging element 531. Meanwhile, for the second light source 32, the light source controller 941 executes PWM control for changing the light emission period vd2 of the excitation light in a state where the maximum light emission period vd max (hereinafter, it is described as a maximum light emission period vd2 max) of the excitation light is larger than the entire line exposure period a (hereinafter, it is described as an entire line exposure period a2) in the second imaging element 532.

That is, the light source controller 941 changes the first light only in a region where the relationship between the exposure area S1 in the first imaging element 531 and the light emission period vd1 of the first light is linear. Meanwhile, the light source controller 941 may change the excitation light in a region where the relationship between the exposure area S2 in the second imaging element 532 and the light emission period vd2 of the excitation light is nonlinear. Note that the exposure area S1 corresponds to a first energy index value according to the present disclosure. The exposure area S2 corresponds to a second energy index value according to the present disclosure.

Therefore, there are the following problems.

Here, it is assumed that the target duty x0 calculated from the brightness of the target light source is 80%. The duty is a rate (%) obtained by dividing the light emission period by the maximum light emission period.

As described above, for the first light, the relationship between the exposure area S1 in the first imaging element 531 and the light emission period vd1 of the first light changes only in a linear region. Therefore, in a case where the light emission period 0.8*vd1_max corresponding to the target duty x0 (80%) is designated and caused to emit light as the light emission period vd1 of the first light, the exposure area S1 is also 80% of the maximum time as illustrated in FIG. 5.

Meanwhile, as described above, for the excitation light, the relationship between the exposure area S2 in the second imaging element 532 and the light emission period vd2 of the excitation light may change in a non-linear region. Therefore, in a case where the light emission period 0.8*vd2_max corresponding to the target duty x0 (80%) is designated as the light emission period vd2 of the excitation light and the excitation light is emitted, as illustrated in FIG. 6, the exposure area S2 does not become 80% of the maximum time.

Here, the exposure areas S1 and S2 correspond to the brightness of the captured image. Therefore, there is a problem that the ratio between the brightness of the normal light image and the brightness of the fluorescence image changes, and the balance of the brightness is deteriorated.

Therefore, the light source controller 941 according to the present disclosure executes the following control in order to maintain the ratio between the brightness of the normal light image and the brightness of the fluorescence image and generate an image suitable for observation in which a balance between the brightness of the normal light image and the brightness of the fluorescence image is favorable.

Here, as illustrated in FIG. 7, it is assumed that imaging is continuously performed (fields (frames) are continuous). In this case, when the light emission timing of the pulsed light is matched with the imaging timing, the preceding and subsequent fields (frames) are also exposed, so that the above-described problem does not occur.

More specifically, when attention is paid to the field (frame) indicated by the solid line in FIG. 7, the reduction in the upper and lower exposure areas Ar1 is compensated by the addition of the exposure area Ar2 by the preceding and subsequent pulsed light. That is, since there is the exposure area Ar1, a region in which the relationship between the light emission period and the exposure area is non-linear does not occur, and the above-described problem does not occur.

Then, in the present embodiment, as illustrated in FIG. 8A, the normal light image is not continuously but intermittently generated by the first imaging element 531, and the fluorescence image is not continuously but intermittently generated by the second imaging element 532, and the state described in FIG. 7 is not obtained, so that the above-described problem occurs.

Furthermore, as illustrated FIG. 8B, even in a case where the imaging element according to the present disclosure includes only one imaging element, the normal light image is generated not continuously but intermittently, and the fluorescence image is generated not continuously but intermittently. Therefore, the state described in FIG. 7 is not obtained, and the above-described problem occurs. This state corresponds to a seventh modification and an eleventh modification described later in which only one imaging element according to the present disclosure is configured.

Note that the above-described problem occurs not only in a case where the normal light image and the fluorescence image are intermittently generated as described above, but also in a case where the timing of the light (pulsed light) emitted from the light source (first and second light sources 31 and 32) is shifted. Then, according to the following control by the light source controller 941 according to the present disclosure, it is also possible to solve the above-described problem caused by the shift of the timing of the light (pulsed light) emitted from the light source (first and second light sources 31 and 32) in this manner.

### Function of Light Source Controller

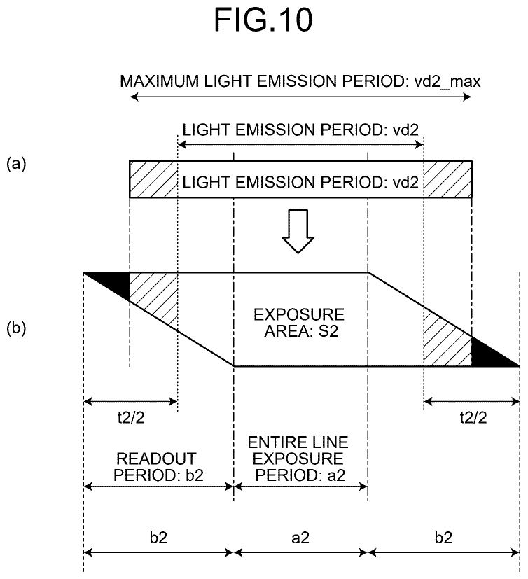

FIG. 9 is a diagram illustrating a calculation expression of the exposure area S1(vd1) in the first imaging element 531. Specifically, FIG. 9 is a diagram corresponding to FIG. 3. FIG. 10 is a diagram illustrating a calculation expression of the exposure area S2(vd2) in the second imaging element 532. Specifically, FIG. 10 is a diagram corresponding to FIG. 3.

Here, the exposure areas S1(vd1) and S2(vd2) in the first and second imaging elements 531 and 532 can be calculated as follows.

Hereinafter, for convenience of calculation, the height (corresponding to current value supplied to first and second light sources 31 and 32) is set to 1 in consideration of the exposure areas S1(vd1) and S2(vd2).

First, a calculation expression of the exposure area S1(vd1) in the first imaging element 531 will be described with reference to FIG. 9.

The exposure area S1(vd1) is a function of the light emission period vd1. As described above, the maximum light emission period vd1_max of the first light is within the entire line exposure period a1. Therefore, the exposure area S1(vd1) can be calculated by the following Expression (1)

$$S 1 ( vd 1 ) = vd 1 * 1 \qquad ( 1 )$$

When the target brightness of the first light source 31 is expressed as a duty x1 with respect to the maximum brightness, x1=S1(vd1)/S1(vd1_max). Note that S1(vd1_max) is an exposure area in the maximum light emission period vd1_max.

Next, a calculation expression of the exposure area S2(vd2) in the second imaging element 532 will be described with reference to FIG. 10.

The exposure area S2(vd2) is a function of the light emission period vd2. As described above, the maximum light emission period vd2_max of the excitation light is larger than the entire line exposure period a2. More specifically, the maximum light emission period vd2_max of the excitation light is larger than the entire line exposure period a2, and is smaller than the entire exposure period (a period obtained by adding the entire line exposure period a2 and two readout periods b2) in the second imaging element 532 for forming a fluorescence image of one field (one frame). That is, in the present embodiment, the entire exposure period in the second imaging element 532 includes a partial period (in FIG. 10, black period) in which the light emission period vd2 of the excitation light cannot be adjusted. Hereinafter, it is assumed that t2=a2+2*b2-vd2 (FIG. 10).

Then, the exposure area S2(vd2) can be calculated by the following Expression (2) in the region of vd2<a2.

$$S 2 ( vd 2 ) = vd 2 * 1 \qquad ( 2 )$$

In addition, the exposure area S2 (vd2) can be calculated by the following Expression (3) in the region of a2<vd2<a2+2*b2.

$$S 2 ( vd 2 ) = a 2 * 1 + 2 ( ( b 2 / 2 ) - ( ( t 2 / 2 ) * ( t 2 / 2 ) / b 2 ) / 2 ) = a 2 * b 2 - ( a 2 + b 2 - vd 2 ) * ( a 2 + b 2 - vd 2 ) / ( 4 * b 2 ) \qquad ( 3 )$$

When the target brightness of the second light source 32 is expressed as a duty x2 with respect to the maximum brightness, x2=S2(vd1)/S2(vd2_max). S2(vd2_max) is an exposure area in the maximum light emission period vd2 max.

Then, the light source controller 941 adjusts the light emission periods vd1 and vd2 as described below.

First, the light source controller 941 calculates a target duty x0 from the brightness of the target light source. Note that the brightness of the target light source may be specified through the input unit 95 (manual dimming) or may be calculated by calculation by the control unit 94 (automatic dimming). Even in the automatic dimming, fine adjustment corresponding to EV correction in the photograph is possible, and the fine adjustment is designated through the input unit 95.

Next, the light source controller 941 calculates the duty x1 in the first light source 31 by the following Expression (4) using the calculated target duty x0.

$$x 0 = x 1 \qquad ( 4 )$$

Next, the light source controller 941 calculates the target exposure area S2_tgt by the following Expression (5) using the calculated target duty x0 and the maximum exposure area S2 (vd2_max).

$$S2\_tgt = S 2 ( vd2\_max ) * x 0 \qquad ( 5 )$$

Next, the light source controller 941 uses the calculated target exposure area S2_tgt and an inverse function of the exposure area S2(vd2) to calculate a light emission period vd2_revised corrected by the following Expression (6).

$$vd2\_revised = inverse function of S 2 ( vd 2 ) ( target exposure area S2\_tgt ) \qquad ( 6 )$$

Here, in a case where the target exposure area S2_tgt is an exposure area within the entire line exposure period a2, the light source controller 941 uses an inverse function of S2(vd2) expressed by Expression (2) in Expression (6). Meanwhile, in a case where the target exposure area S2_tgt is the exposure area exceeding the entire line exposure period a2, the light source controller 941 uses the inverse function of S2(vd2) expressed by Expression (3) in Expression (6).

Next, the light source controller 941 calculates a duty x2_revised corrected by the following Expression (7) using the calculated light emission period vd2_revised and the maximum light emission period vd2_max.

$$x2\_revised = vd2\_revised / vd2\_max \qquad ( 7 )$$

That is, the above-described problem that occurs when x2=x1 is corrected by calculation, and x2 revised is obtained. In other words, the light source controller 941 adjusts the light emission periods vd1 and vd2 so that the ratio between the exposure area S1 and the exposure area S2 becomes a predetermined ratio. That is, the light source controller 941 adjusts the light emission periods vd1 and vd2 so that the energy of exposing the first return light in the first imaging element 531 and the energy of exposing the second return light in the second imaging element 532 have a predetermined ratio. In a case where the light emission periods vd1 and vd2 are adjusted in this manner, there is a case where the rate of change in the light emission period vd1 of the first light and the rate of change in the light emission period vd2 of the excitation light are different in the non-linear region illustrated in FIG. 6.

Then, the light source controller 941 causes the first light source 31 to emit the first light in the light emission period vd1 corresponding to the duty x1 from the first light source 31 by controlling the first light source 31 with the duty x1. Furthermore, the light source controller 941 controls the second light source 32 with the corrected duty x2 to cause the second light source 32 to emit excitation light in the corrected light emission period vd2_revised. That is, the light source controller 941 alternately and sequentially turns on the first and second light sources 31 and 32.

According to the present embodiment described above, the following effects are obtained.

In the control device 9 according to the present embodiment, the light source controller 941 adjusts the light emission periods vd1 and vd2 so that the ratio between the exposure area S1 and the exposure area S2 becomes a predetermined ratio while making the light emission period vd2 of the excitation light adjustable to be larger than the entire line exposure period a2. That is, the light emission periods vd1 and vd2 are adjusted while maintaining the ratio between the brightness of the normal light image and the brightness of the fluorescence image.

Therefore, according to the control device 9 of the present embodiment, it is possible to maintain the ratio between the brightness of the normal light image and the brightness of the fluorescence image, and generate the image suitable for observation in which the balance between the brightness of the normal light image and the brightness of the fluorescence image is favorable.

In addition, the light source controller 941 calculates the duties x1 and x2 from the target duty x0 using the arithmetic expressions (4) and (5) to (7). Therefore, the light emission periods vd1 and vd2 can be adjusted by simple processing.

In addition, in Expression (6), the light source controller 941 uses an arithmetic expression different between a case where the target exposure area S2_tgt is an exposure area within the entire line exposure period a2 and a case where the target exposure area S2_tgt is an exposure area exceeding the entire line exposure period a2. Therefore, the above-described effect that “it is possible to maintain the ratio between the brightness of the normal light image and the brightness of the fluorescence image and to generate an image suitable for observation in which the balance between the brightness of the normal light image and the brightness of the fluorescence image is favorable” can be suitably realized.

### OTHER EMBODIMENTS

Although the embodiments for carrying out the present disclosure have been described so far, the present disclosure should not be limited only by the above-described embodiments.

In the above-described embodiment, first to twelfth modifications described below may be adopted.

### First Modification

In the above-described embodiment, other configurations may be adopted as long as at least one of the first and second imaging elements 531 and 532 has a region in which the relationship between the exposure area and the light emission period is nonlinear.

For example, in the above-described embodiment, a charge coupled device (CCD) that is a global shutter type imaging element may be adopted as one of the first and second imaging elements 531 and 532.

Furthermore, for example, in the above-described embodiment, the entire line exposure period of at least one of the first and second imaging elements 531 and 532 may be set to 0. That is, at least one of the first and second imaging elements 531 and 532 does not have a linear relationship between the exposure period S and the light emission period vd, and has only a non-linear relationship.

Moreover, for example, in the above-described embodiment, at least one of the first and second imaging elements 531 and 532 may employ an imaging element having a region that is different from the rolling shutter type and has a non-linear relationship between the exposure area and the light emission period.

### Second Modification

In the above-described embodiment, the maximum light emission period vd1_max of the first light may be made larger than the entire line exposure period a1, similarly to the maximum light emission period vd2 max of the excitation light. More specifically, the maximum light emission period vd1_max of the first light is larger than the entire line exposure period a1 and smaller than the entire exposure period (a period obtained by adding the entire line exposure period a1 and the two readout periods b1) in the first imaging element 531 for forming a normal light image of one field (one frame), that is, in the present modification, the entire exposure period in the first imaging element 531 includes a partial period (in FIG. 9, black period) in which the light emission period vd1 of the first light is non-adjustable. Hereinafter, t1=a1+2*b1−vd1 (FIG. 9).

Then, the exposure area S1(vd1) can be calculated by Expression (1) in the region of vd1<a1.

In addition, the exposure area S1(vd1) can be calculated by the following Expression (8) in the region of a1<vd1<a1+2*b1.

$$S 1 ( vd 1 ) = a 1 * 1 + 2 ( ( b 1 / 2 ) - ( ( t 1 / 2 ) ⋆ ( t 1 / 2 ) / b 1 ) / 2 ) = a 1 * b 1 - ( a 1 + b 1 - vd 1 ) ⋆ ( a 1 + b 1 - vd 1 ) / ( 4 / b 1 ) \qquad ( 8 )$$

Then, the light source controller 941 adjusts the light emission period vd1 as described below. Note that adjustment of the light emission period vd2 is similar to that of the above-described embodiment.

First, the light source controller 941 calculates the target exposure area S1_tgt by the following Expression (9) using the calculated target duty x0 and the maximum exposure area S1 (vd1_max).

$$S1\_tgt = S 1 ( vd1\_max ) * x 0 \qquad ( 9 )$$

Next, the light source controller 941 uses the calculated target exposure area S1_tgt and an inverse function of the exposure area S1(vd1) to calculate the light emission period vd1_revised corrected by the following Expression (10).

$$vd1\_revised = inverse function of S 1 ( vd 1 ) ( target exposure area S1\_tgt ) \qquad ( 10 )$$

Here, in a case where the target exposure area S1_tgt is an exposure area within the entire line exposure period a1, the light source controller 941 uses an inverse function of S1(vd1) expressed by Expression (1) in Expression (10). Meanwhile, in a case where the target exposure area S1_tgt is the exposure area exceeding the entire line exposure period a1, the light source controller 941 uses the inverse function of S1(vd1) expressed by Expression (8) in Expression (10).

Next, the light source controller 941 calculates the duty x1_revised corrected by the following Expression (11) using the calculated light emission period vd1 revised and the maximum light emission period vd1_max.

$$x 1 \_revised = vd1\_revised / vd1\_max \qquad ( 11 )$$

Then, the light source controller 941 causes the first light source 31 to emit the first light in the corrected light emission period vd1_revised from the first light source 31 by controlling the first light source 31 with the corrected duty x1 revised.

### Third Modification

In the above-described embodiment, the light source controller 941 calculates the duties x1 and x2 from the target duty x0 using the arithmetic expressions (4) and (5) to (7), but the present disclosure is not limited thereto, and the duties x1 and x2 corresponding to the target duty x0 may be specified with reference to a table. At this time, when the duties x1 and x2 corresponding to the target duty x0 are not in the table, the duties x1 and x2 may be calculated by interpolation.

### Fourth Modification

In the above-described embodiment, when the exposure areas S1(vd1) and S2(vd2) are calculated, all the weights of the entire surface are the same, but the present disclosure is not limited thereto. That is, the exposure areas S1(vd1) and S2(vd2) may be calculated by performing different weights at the portions to be exposed.

For example, the exposure areas S1(vd1) and S2(vd2) may be calculated by calculation in which the weights of the upper, lower, left, and right portions of the exposure surface are reduced and the brightness of the central portion is regarded as important.

Hereinafter, the exposure area S2 (vd2) will be described as an example.

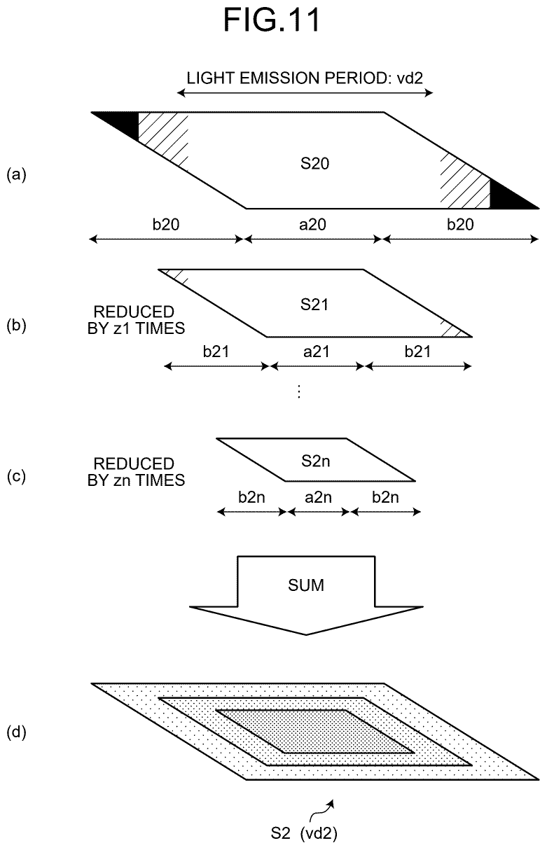

FIG. 11 is a diagram for describing a fourth modification of the embodiment. Specifically, FIG. 11 is a diagram corresponding to (b) of FIG. 3.

Note that the light emission period vd2 is common to all the parallelograms illustrated in FIG. 11. In addition, the calculation function of each exposure area according to each parallelogram illustrated in FIG. 11 is common by using the entire line exposure period and the readout period as arguments.

Here, the original exposure area is defined as an exposure area S20 ((a) of FIG. 11). The exposure area S20 can be expressed as S20=S(a20, b20, vd2) as a function of the entire line exposure period a2 (hereinafter, it is described as an entire line exposure period a20), the readout period b2 (hereinafter, it is described as a reading period b20), and the light emission period vd2.

Furthermore, a parallelogram in which length and width are reduced by z1 times with respect to the exposure area S20 is considered ((b) of FIG. 11), and similarly, when this exposure area is the exposure area S21, it can be expressed as S21=S(a21, b21, vd2)*z1.

Similarly, when the exposure area of the parallelogram reduced by zn times in the nth work is defined as an exposure area S2n ((c) of FIG. 11), S2n=S(a2n, b2n, Vd2)*zn can be expressed. Note that vd2>a2n+2*b2n.

Then, as illustrated in the following Expression (12), a sum of the above-described exposure areas is defined as an exposure area S2(vd2) ((d) in FIG. 11).

$$S 2 ( vd 2 ) = {\sum }_{n = 0}^{N} S ( a 2 n , b 2 n , vd 2 ) * zn \qquad ( 12 )$$

### Fifth Modification

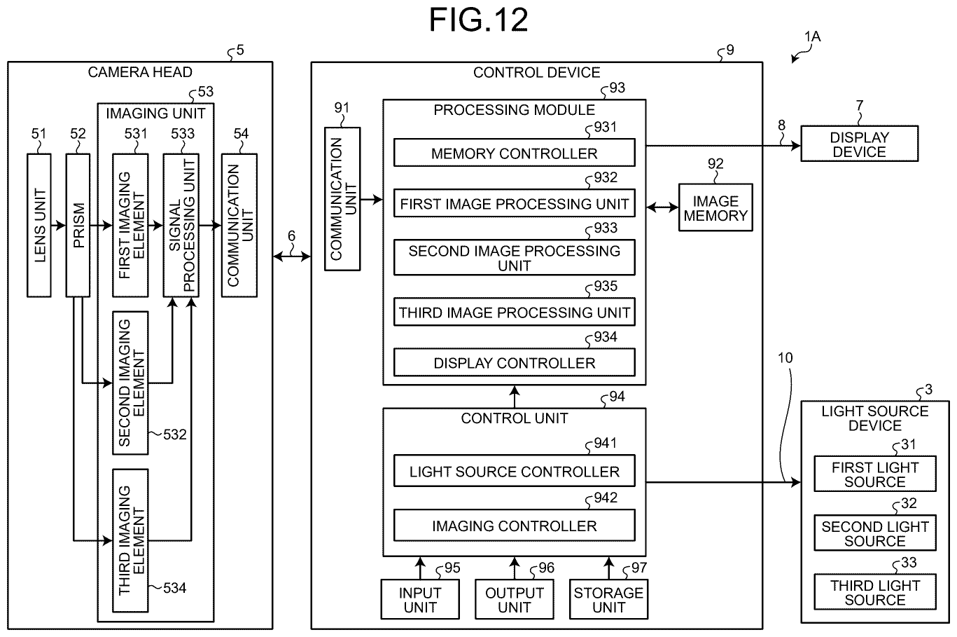

FIG. 12 is a diagram for describing a fifth modification of the embodiment. Specifically, FIG. 12 is a diagram corresponding to FIG. 2.

In the above-described embodiment, the two light sources of the first and second light sources 31 and 32 and the two imaging elements of the first and second imaging elements 531 and 532 are adopted, but the present disclosure is not limited thereto. For example, three or more light sources and three or more imaging elements may be adopted.

Note that FIG. 12 illustrates a configuration in which three light sources and three imaging elements are employed.

Specifically, in a medical observation system 1A according to the present fifth modification, as illustrated in FIG. 12, in the imaging unit 53, a third imaging element 534 is added as a third imaging element in addition to the first and second imaging elements 531 and 532. Furthermore, in addition to the first and second image processing units 932 and 933, a third image processing unit 935 is added to the processing module 93 as a third image processing unit. Furthermore, in the light source device 3, in addition to the first and second light sources 31 and 32, a third light source 33 is added as a third light source.

For example, the first light source 31 emits first light such as white light similarly to the above-described embodiment. The second light source 32 emits infrared light. The third light source 33 emits ultraviolet light.

Here, the prism 52 separates the return light of the first light from the observation target, the return light of the infrared light from the observation target, and the return light of the ultraviolet light from the observation target. Then, the first imaging element 531 images the return light of the first light. The second imaging element 532 images the return light of infrared light. The third imaging element 534 images the return light of ultraviolet light. Hereinafter, a captured image generated by imaging of the first imaging element 531 will be referred to as a first captured image. Furthermore, a captured image generated by imaging of the second imaging element 532 will be referred to as a second captured image. Furthermore, a captured image generated by imaging of the third imaging element 534 is referred to as a third captured image.

Here, the memory controller 931 controls writing of the captured image to the image memory 92 and reading of the captured image from the image memory 92 to input the first captured image to the first image processing unit 932, to input the second captured image to the second image processing unit 933, and to input the third captured image to the third image processing unit 935. The first image processing unit 932 executes first image processing on the input first captured image. The second image processing unit 933 executes second image processing on the input second captured image. The third image processing unit 935 executes third image processing on the input third captured image. Then, under the control of the control unit 94, the display controller 934 generates a video signal for displaying at least one of the first captured image after the first image processing is executed by the first image processing unit 932, the second captured image after the second image processing is executed by the second image processing unit 933, and the third captured image after the third image processing is executed by the third image processing unit 935.

Note that, also in the present fifth modification, the first imaging element 531 generates the first captured image not continuously but intermittently. Furthermore, the second imaging element 532 generates the second captured image not continuously but intermittently. Further, the third imaging element 534 generates the third captured image not continuously but intermittently.

### Sixth Modification

In the above-described embodiment, the light source and the imaging element correspond to each other on a one-to-one basis, but the present disclosure is not limited thereto. For example, a configuration in which a plurality of imaging elements corresponds to one light source may be adopted. As a specific example, one light source that emits xenon light is used, and white light imaging and ultraviolet light imaging are executed by two imaging elements.

### Seventh Modification

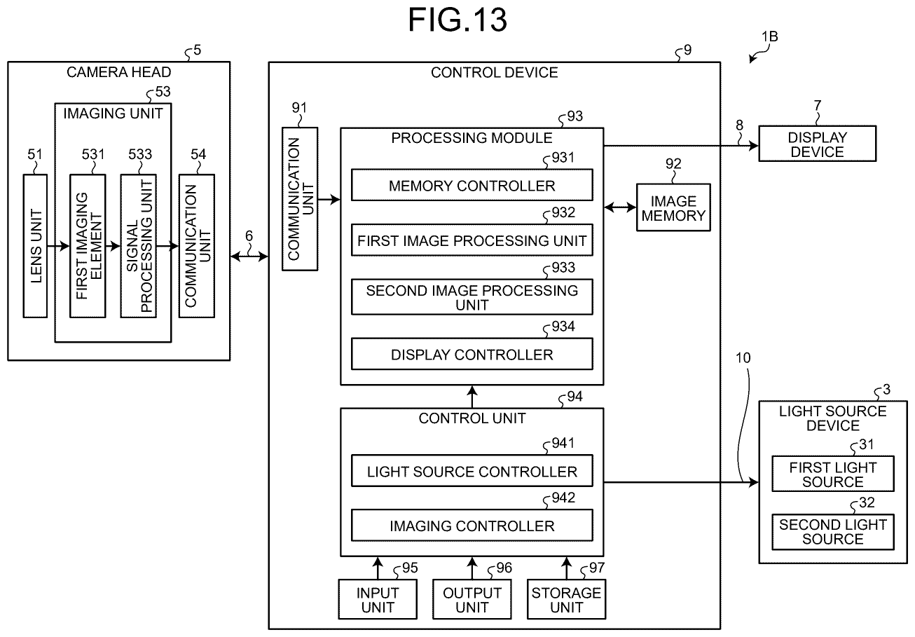

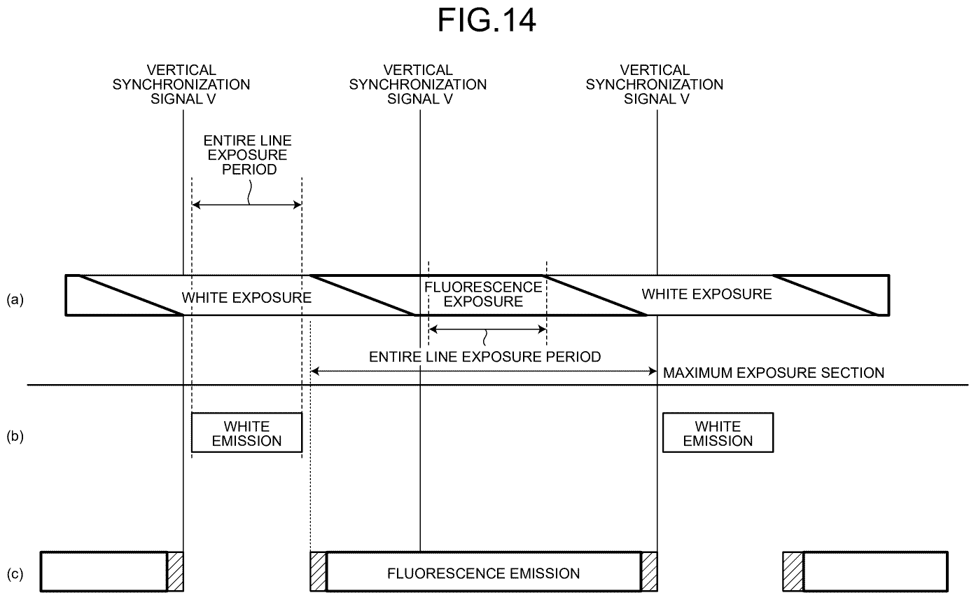

FIGS. 13 and 14 are diagrams for describing a seventh modification of the embodiment. Specifically, FIG. 13 is a diagram corresponding to FIG. 2. (a) of FIG. 14 corresponds to part (b) of FIG. 3. (b) of FIG. 14 corresponds to part (a) of FIG. 9. (c) of FIG. 14 corresponds to part (a) of FIG. 10.

In the above-described embodiment, the light source and the imaging element correspond to each other on a one-to-one basis, but the present disclosure is not limited thereto. For example, a configuration may be adopted in which one imaging element corresponds to a plurality of light sources, and the one imaging element is driven in a time division manner to generate a plurality of captured images.

Note that FIG. 13 illustrates a configuration in which two light sources and one imaging element are employed.

Specifically, in a medical observation system 1B according to the seventh modification, as illustrated in FIG. 13, the prism 52 is omitted in the camera head 5. Furthermore, the second imaging element 532 is omitted from the imaging unit 53.

The control of the first and second light sources 31 and 32 by the light source controller 941 is similar to that of the above-described embodiment as illustrated in (b) of FIG. 14 and (c) of FIG. 14.

Then, the first imaging element 531 generates the normal light image and the fluorescence image in a time division manner as illustrated in (a) of FIG. 14 under the control of the imaging controller 942.

### Eighth Modification

In the above-described embodiment, the light source controller 941 controls the first and second light sources 31 and 32 by the duties x1 and x2, but the present disclosure is not limited thereto, and the first and second light sources 31 and 32 may be controlled by the light emission periods vd1 and vd2 revised.

### Ninth Modification

In the above-described embodiment, the light source controller 941 may change the parameters (a1, a2, b1, b2) of the arithmetic expressions (4) and (5) to (7) for calculating the duties x1 and x2 from the target duty x0 according to the type (for example, a vertical synchronization signal of the NTSC system, a vertical synchronization signal of the PAL system, and the like) of the vertical synchronization signal.

### Tenth Modification

In the above-described embodiment, the light source controller 941 may switch the arithmetic expressions (4), (5) to (7) for calculating the duties x1 and x2 from the target duty x0 according to at least one (for example, a difference in the length of the entire exposure period, a difference in the length of the entire line exposure period, normal exposure, long exposure, and the like) of the type (the type of the imaging element) of the camera head 5 and the operation control (for example, normal exposure, long time exposure, and the like) of the camera head 5. The same applies to the above-described third modification using a table instead of the arithmetic expression.

### Eleventh Modification

In the above-described embodiment, a configuration using one light source and one imaging element may be adopted. For example, in the above-described embodiment, the prism 52, the first light source 31, and the first imaging element 531 may be omitted.

That is, in the present eleventh modification, the light source controller 941 adjusts the light emission period vd2 of the excitation light based on the first energy index value (exposure area S2(vd2)) which is an energy index value serving as an index of energy of light obtained by exposing the second return light of the excitation light (corresponding to the first light according to the present disclosure) in the second imaging element 532.

### Twelfth Modification

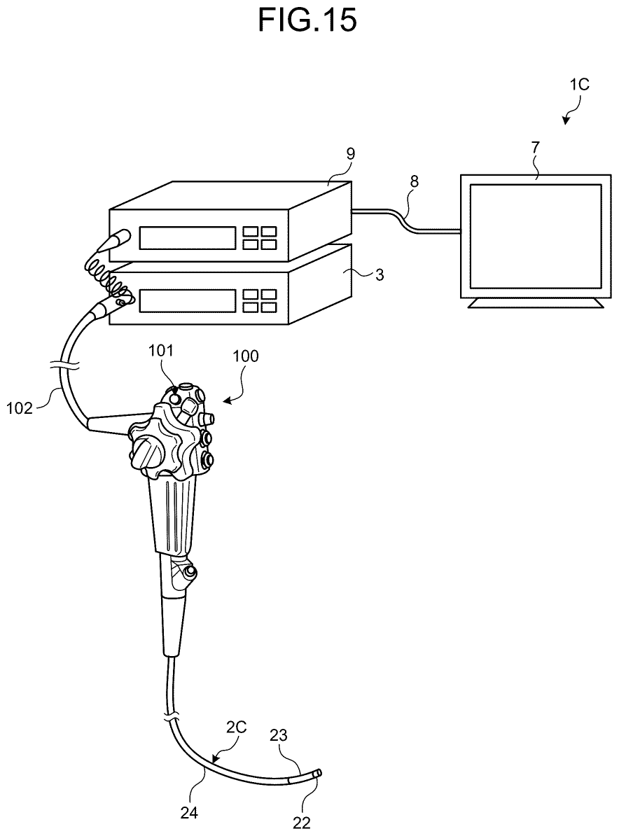

FIG. 15 is a diagram for describing a twelfth of the embodiment.

A medical observation system 1C according to the present twelfth modification is a medical observation system using a so-called video scope (flexible endoscope) having an imaging unit on the distal end side of the insertion unit.

As illustrated in FIG. 15, the medical observation system 1C includes an endoscope 100 that images an in-vivo image of an observed region by inserting an insertion unit 2C into a living body and outputs a captured image, a light source device 3 that emits first light and excitation light from a distal end of the endoscope 100, a control device 9 that processes the captured image output from the endoscope 100, and a display device 7 that is connected to the control device 9 via a second transmission cable 8 and displays an image based on a video signal processed by the control device 9.

As illustrated in FIG. 15, the endoscope 100 includes an insertion unit 2C having a flexible elongated shape, an operating unit 101 connected to a proximal end side of the insertion unit 2C and receiving various operations, and a universal cord 102 extending in a direction different from a direction in which the insertion unit 2C extends from the operating unit 101 and incorporating various cables connected to the light source device 3 and the control device 9.

As illustrated in FIG. 15, the insertion unit 2C includes a distal end portion 22, a bendable bending portion 23 connected to the proximal end side of the distal end portion 22 and configured by a plurality of bending pieces, and an elongated flexible tube portion 24 connected to the proximal end side of the bending portion 23 and having flexibility.

Although not specifically illustrated, substantially the same configuration as the camera head 5 described in the above-described embodiment is incorporated in the distal end portion 22. Then, the captured image captured by the distal end portion 22 (imaging element) is output to the control device 9 via the operating unit 101 and the universal cord 102.

### Thirteenth Modification

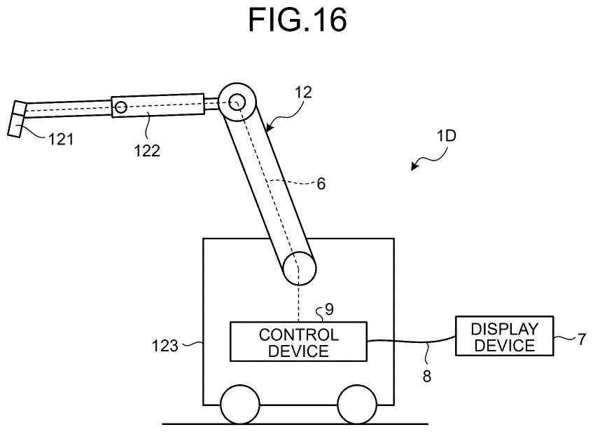

FIG. 16 is a diagram for describing a thirteenth modification of the embodiment.

A medical observation system 1D according to the present thirteenth modification is a medical observation system using a surgical microscope that enlarges and images a predetermined field of view of an inside of a subject (inside of a living body) which is the observation target or a surface of the subject (surface of the living body).

As illustrated in FIG. 16, the medical observation system 1D includes a surgical microscope 12 that images an image for observing the subject and outputs the captured image, a control device 9 that processes the captured image output from the surgical microscope 12, and a display device 7 that is connected to the control device 9 via a second transmission cable 8 and displays an image based on a video signal processed by the control device 9.

As illustrated in FIG. 16, the surgical microscope 12 includes a microscope unit 121 that enlarges and captures a minute portion of the subject and outputs the captured image, a support unit 122 that is connected to a proximal end portion of the microscope unit 121 and includes an arm that rotatably supports the microscope unit 121, and a base unit 123 that rotatably holds a proximal end portion of the support unit 122 and is movable on a floor surface.

Then, as illustrated in FIG. 16, the control device 9 is installed on the base unit 123. In addition, although not specifically illustrated, the light source device 3 that emits the first light and the excitation light from the surgical microscope 12 to the observation target is also installed in the base unit 123.

Note that the base unit 123 may be fixed to a ceiling, a wall surface, or the like to support the support unit 122, instead of being movably provided on the floor surface.

Although not specifically illustrated, the microscope unit 121 incorporates substantially the same configuration as the camera head 5 described in the above-described embodiment. Then, the captured image captured by the microscope unit 121 (imaging element) is output to the control device 9 via the first transmission cable 6 wired along the support unit 122.

### Fourteenth Modification

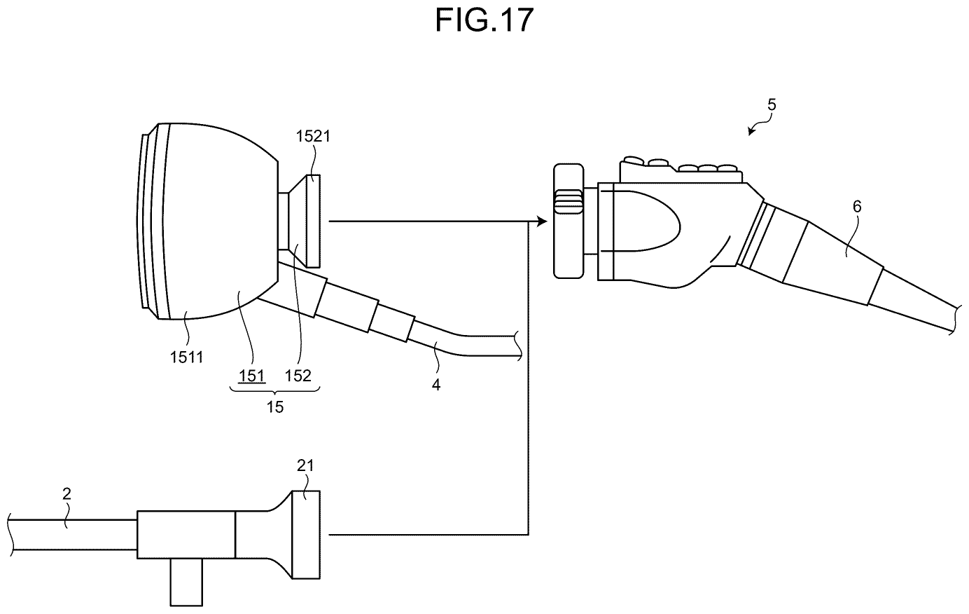

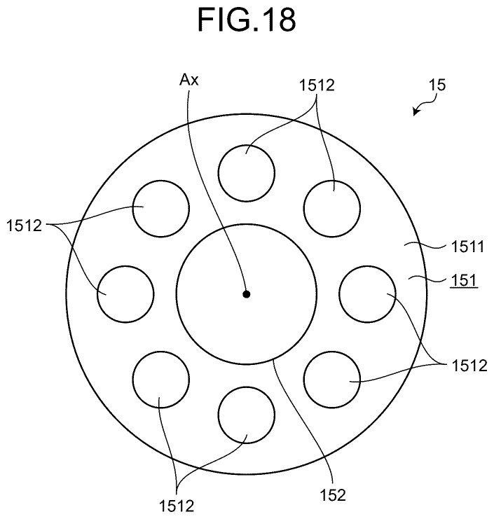

FIGS. 17 and 18 are diagrams for describing a fourteenth modification of the embodiment. Specifically, FIG. 17 is a side view of a ring light 15. FIG. 18 is a view of the ring light 15 as viewed from the front side (left side in FIG. 17).

In the present fourteenth modification, in addition to the insertion unit 2 described in the above-described embodiment, the ring light 15 illustrated in FIGS. 17 and 18 is detachably connected to the camera head 5. That is, as illustrated in FIG. 17, the insertion unit 2 may be connected to the camera head 5 or the ring light 15 may be connected to the camera head 5 depending on the use state of the user.

The ring light 15 is not inserted into the observation target like the insertion unit 2, supplies the first light and the excitation light to a surgical site, and takes in the return light of the first light and the excitation light from the surgical site. As illustrated in FIGS. 17 and 18, the ring light 15 includes an illumination unit 151 and a subject image capturing unit 152 that captures a subject image.

As illustrated in FIGS. 17 and 18, the illumination unit 151 includes a casing 1511 and a plurality of illumination lenses 1512.

The casing 1511 has an annular shape centered on an optical axis Ax. The other end of the light guide 4 is detachably connected to the casing 1511.

As illustrated in FIG. 18, the plurality of illumination lenses 1512 are arranged at predetermined intervals along a circumferential direction centered on the optical axis Ax on the end surface on the front side of the casing 1511. Then, the plurality of illumination lenses 1512 irradiate the surgical site with the first light and the excitation light supplied from the light source device 3 and introduced into the casing 1511 via the light guide 4.

The subject image capturing unit 152 extends along the optical axis Ax. Furthermore, in the subject image capturing unit 152, an optical system configured using one or a plurality of lenses and configured to collect the return light of the first light and the excitation light emitted from the plurality of illumination lenses 1512 and passing through the surgical site is provided. Furthermore, a connection unit 1521 is provided at an end portion on a proximal end side (In FIG. 17, right side) of the subject image capturing unit 152. The connection unit 1521 is designed (shaped) to be compatible with the eyepiece unit 21 in the insertion unit 2, and is detachably connected to the camera head 5.

Note that the following configurations also belong to the technical scope of the present disclosure.
(1) A medical observation system includes: a light source device that emits first light and second light to an observation target; an imaging device that images return light of the first light and the second light from the observation target, and changes the number of horizontal lines to be simultaneously exposed by at least one of the return light of the first light and the return light of the second light; and a light source controller that adjusts light emission periods of the first light and the second light such that energy of light obtained by exposing the return light of the first light in the imaging device and energy of light obtained by exposing the return light of the second light in the imaging device have a predetermined ratio.
(2) The medical observation system according to (1), in which the imaging device intermittently images the return light of the first light and the return light of the second light.
(3) The medical observation system according to (1) or (2), in which when a value obtained by adding exposure periods for the respective horizontal lines in the imaging device is defined as an energy index value, the imaging device includes a region in which a relationship between the energy index value and an exposure period of light is nonlinear, and the light source controller controls the light emission periods of the first light and the second light are adjusted such that a first energy index value related to the imaging of the return light of the first light and a second energy index value related to the imaging of the return light of the second light have a predetermined ratio.
(4) The medical observation system according to any one of (1) to (3), in which the light source controller adjusts the light emission periods of the first light and the second light using a predetermined arithmetic expression.
(5) The medical observation system according to (4), in which the imaging device includes an imaging element that is a rolling shutter type in which a plurality of pixels are two-dimensionally arranged in units of horizontal lines and has an entire line exposure period in which all the horizontal lines in an effective pixel region are simultaneously exposed, and the light source controller uses the arithmetic expression different between a case of adjusting a light emission period of at least one of the first light and the second light within the entire line exposure period and a case of adjusting a light emission period of at least one of the first light and the second light outside the entire line exposure period.
(6) The medical observation system according to (4) or (5), in which the imaging device includes a region in which a relationship between the energy index value serving as an index of energy of exposed light and an exposure period of the light is linear and a region in which the relationship between the energy index value and the exposure period of the light is nonlinear, and the light source controller uses the arithmetic expression different between a case where the light emission period of at least one of the first light and the second light is adjusted in the linear region and a case where the light emission period of the at least one of the first light and the second light is adjusted in the non-linear region.
(7) The medical observation system according to any one of (4) to (6), in which the light source controller makes the light emission periods of the first light and the second light non-adjustable in a partial period of an entire exposure period in the imaging device for forming an image of one frame, and the arithmetic expression is an arithmetic expression that does not take into consideration the partial period in which the light emission periods of the first light and the second light are non-adjustable.
(8) The medical observation system according to any one of (4) to (7), in which the light source controller changes a parameter of the arithmetic expression according to a type of a vertical synchronization signal.
(9) The medical observation system according to any one of (4) to (8), in which the light source controller switches the arithmetic expression according to at least one of a type of the imaging device and operation control of the imaging device.
(10) The medical observation system according to any one of (1) to (9), in which the imaging device includes a region in which a relationship between an energy index value serving as an index of energy of exposed light and an exposure period of the light is linear and a region in which the relationship between the energy index value and the exposure period of the light is nonlinear, the first light is light including at least a part of a visible light wavelength band, the second light is excitation light for exciting a substance contained in the observation target, and the light source controller makes a light emission period of the first light in the linear region adjustable, and makes a light emission period of the second light in the non-linear region adjustable.
(11) The medical observation system according to any one of (1) to (10), in which a rate of change in a light emission period of the first light is sometimes different from a rate of change in a light emission period of the second light.
(12) The medical observation system according to any one of (1) to (3), in which the light source controller adjusts light emission periods of the first light and the second light using a predetermined table.
(13) The medical observation system according to (12), in which the light source controller switches the table according to at least one of a type of the imaging device and operation control of the imaging device.
(14) The medical observation system according to (3), in which the light source controller calculates at least one of the first energy index value and the second energy index value by performing different weighting at a portion where at least one of return light of the first light and return light of the second light is exposed in the imaging device.
(15) A medical observation system includes: a light source device that emits light to an observation target; an imaging device that images return light of the light from the observation target and changes the number of horizontal lines to be simultaneously exposed by the return light of the light; and a light source controller that adjusts a light emission period of the light based on an energy index value serving as an index of a target value of energy of the return light exposed in the imaging device.
(16) The medical observation system according to any one of (1) to (15), in which the light source device includes a plurality of light sources.
(17) The medical observation system according to (16), in which the imaging device includes only one imaging element.
(18) The medical observation system according to any one of (1) to (16), in which the imaging device includes a plurality of imaging elements.
(19) The medical observation system according to any one of (1) to (18), in which the imaging device includes a second imaging element that images return light of the second light, and the second imaging element does not have a relationship in which a relationship between a second energy index value serving as an index of energy of light of the return light of the exposed second light and an exposure period of the second light is linear, and has only a non-linear region.
(20) The medical observation system according to any one of (1) to (19), in which the imaging device includes a first imaging element that images the return light of the first light, and a second imaging element that images the return light of the second light, and each of both the first imaging element and the second imaging element has a region in which a relationship between an energy index value serving as an index of energy of the exposed light and an exposure period of the light is nonlinear.
(21) A light source control device includes: a light source controller that controls an operation of a light source device and causes the light source device to emit first light and second light to an observation target, in which each return light of the first light and the second light from the observation target is imaged by an imaging device that changes the number of horizontal lines simultaneously exposed by at least one of the return light of the first light and the return light of the second light, and the light source controller adjusts light emission periods of the first light and the second light such that energy of light obtained by exposing the return light of the first light in the imaging device and energy of light obtained by exposing the return light of the second light in the imaging device have a predetermined ratio.
(22) A light source control device includes: a light source controller that controls an operation of a light source device and causes the light source device to emit light to an observation target, in which return light of the light from the observation target is imaged by an imaging device that changes the number of horizontal lines simultaneously exposed by the return light, and the light source controller adjusts a light emission period of the light based on an energy index value serving as an index of a target value of energy of the return light exposed in the imaging device.

According to the medical observation system and the light source control device according to the present disclosure, it is possible to generate an image suitable for observation.

Although the disclosure has been described with respect to specific embodiments for a complete and clear disclosure, the appended claims are not to be thus limited but are to be construed as embodying all modifications and alternative constructions that may occur to one skilled in the art that fairly fall within the basic teaching herein set forth.

What is claimed is:

1. A medical observation system comprising:
a light source device configured to emit first light and second light to an observation target;
an imaging device configured to
image return light of the first light and the second light from the observation target, and
change number of horizontal lines to be simultaneously exposed by at least one of the return light of the first light and the return light of the second light; and
a light source controller configured to adjust light emission periods of the first light and the second light such that energy of light obtained by exposing the return light of the first light in the imaging device and energy of light obtained by exposing the return light of the second light in the imaging device have a predetermined ratio.

2. The medical observation system according to claim 1, wherein the imaging device is configured to intermittently image the return light of the first light and the return light of the second light.

3. The medical observation system according to claim 1, wherein
when a value obtained by adding exposure periods for the respective horizontal lines in the imaging device is defined as an energy index value,
the imaging device includes a region in which a relationship between the energy index value and an exposure period of light is nonlinear, and
the light source controller is configured to control the light emission periods of the first light and the second light are adjusted such that a first energy index value related to the imaging of the return light of the first light and a second energy index value related to the imaging of the return light of the second light have a predetermined ratio.

4. The medical observation system according to claim 1, wherein the light source controller is configured to adjust the light emission periods of the first light and the second light using a predetermined arithmetic expression.

5. The medical observation system according to claim 4, wherein the imaging device includes an imaging element that is a rolling shutter type in which a plurality of pixels are two-dimensionally arranged in units of horizontal lines and has an entire line exposure period in which all the horizontal lines in an effective pixel region are simultaneously exposed, and
the light source controller is configured to use the arithmetic expression different between a case of adjusting a light emission period of at least one of the first light and the second light within the entire line exposure period and a case of adjusting a light emission period of at least one of the first light and the second light outside the entire line exposure period.

6. The medical observation system according to claim 4, wherein
the imaging device includes a region in which a relationship between an energy index value serving as an index of energy of exposed light and an exposure period of the light is linear and a region in which the relationship between the energy index value and the exposure period of the light is nonlinear, and
the light source controller is configured to use the arithmetic expression different between a case where the light emission period of at least one of the first light and the second light is adjusted in the linear region and a case where the light emission period of the at least one of the first light and the second light is adjusted in the non-linear region.

7. The medical observation system according to claim 4, wherein
the light source controller is configured to make the light emission periods of the first light and the second light non-adjustable in a partial period of an entire exposure period in the imaging device for forming an image of one frame, and
the arithmetic expression is an arithmetic expression that does not take into consideration the partial period in which the light emission periods of the first light and the second light are non-adjustable.

8. The medical observation system according to claim 4, wherein the light source controller is configured to change a parameter of the arithmetic expression according to a type of a vertical synchronization signal.

9. The medical observation system according to claim 4, wherein the light source controller is configured to switch the arithmetic expression according to at least one of a type of the imaging device and operation control of the imaging device.

10. The medical observation system according to claim 1, wherein
the imaging device includes a region in which a relationship between an energy index value serving as an index of energy of exposed light and an exposure period of the light is linear and a region in which the relationship between the energy index value and the exposure period of the light is nonlinear,
the first light is light including at least a part of a visible light wavelength band,
the second light is excitation light for exciting a substance contained in the observation target, and
the light source controller is configured to
make a light emission period of the first light in the linear region adjustable, and
make a light emission period of the second light in the non-linear region adjustable.

11. The medical observation system according to claim 1, wherein a rate of change in a light emission period of the first light is sometimes different from a rate of change in a light emission period of the second light.

12. The medical observation system according to claim 1, wherein the light source controller is configured to adjust light emission periods of the first light and the second light using a predetermined table.

13. The medical observation system according to claim 12, wherein the light source controller is configured to switch the table according to at least one of a type of the imaging device and operation control of the imaging device.

14. The medical observation system according to claim 3, wherein the light source controller is configured to calculate at least one of the first energy index value and the second energy index value by performing different weighting at a portion where at least one of return light of the first light and return light of the second light is exposed in the imaging device.

15. A medical observation system comprising:
a light source device configured to emit light to an observation target;
an imaging device configured to
image return light of the light from the observation target, and
change number of horizontal lines to be simultaneously exposed by the return light of the light; and
a light source controller configured to adjust a light emission period of the light based on an energy index value serving as an index of a target value of energy of the return light exposed in the imaging device.

16. The medical observation system according to claim 1, wherein the light source device includes a plurality of light sources.

17. The medical observation system according to claim 16, wherein the imaging device includes only one imaging element.

18. The medical observation system according to claim 1, wherein the imaging device includes a plurality of imaging elements.

19. The medical observation system according to claim 1, wherein
the imaging device includes a second imaging element configured to image return light of the second light, and
the second imaging element does not have a relationship in which a relationship between a second energy index value serving as an index of energy of light of the return light of the exposed second light and an exposure period of the second light is linear, and has only a non-linear region.

20. The medical observation system according to claim 1, wherein the imaging device includes
a first imaging element configured to image the return light of the first light, and
a second imaging element configured to image the return light of the second light, and
each of both the first imaging element and the second imaging element has a region in which a relationship between an energy index value serving as an index of energy of the exposed light and an exposure period of the light is nonlinear.

21. A light source control device comprising
a light source controller configured to
control an operation of a light source device, and
cause the light source device to emit first light and second light to an observation target, wherein
each return light of the first light and the second light from the observation target is imaged by an imaging device that changes the number of horizontal lines simultaneously exposed by at least one of the return light of the first light and the return light of the second light, and
the light source controller is configured to adjust light emission periods of the first light and the second light such that energy of light obtained by exposing the return light of the first light in the imaging device and energy of light obtained by exposing the return light of the second light in the imaging device have a predetermined ratio.

22. A light source control device comprising
a light source controller is configured to
control an operation of a light source device, and
cause the light source device to emit light to an observation target, wherein
return light of the light from the observation target is imaged by an imaging device that changes the number of horizontal lines simultaneously exposed by the return light, and
the light source controller is configured to adjust a light emission period of the light based on an energy index value serving as an index of a target value of energy of the return light exposed in the imaging device.
